(19) United States
(12) Patent Application Publication
(10) Pub. No.: US 2014/0249449 A1
(43) Pub. Date: Sep. 4, 2014

(54) SYSTEM FOR COLLECTING AT LEAST ONE SAMPLE OF ANIMAL TISSUE AND CORRESPONDING SAMPLING DEVICE, STORAGE DEVICE, AND MANUFACTURING METHOD

(71) Applicants: Jean-Jacques Hilpert, Vitre (FR); Jean-Jacques Destoumieux, Lescure D'Albigeois (FR); Bruno Teychene, Mouzieys-Teulet (FR); Johan Decaluwe, Laval (FR)

(72) Inventors: Jean-Jacques Hilpert, Vitre (FR); Jean-Jacques Destoumieux, Lescure D'Albigeois (FR); Bruno Teychene, Mouzieys-Teulet (FR); Johan Decaluwe, Laval (FR)

(73) Assignee: Allflex Europe, Vitre (FR)

(21) Appl. No.: 14/233,824

(22) PCT Filed: Jul. 17, 2012
(86) PCT No.: PCT/EP2012/064018

§ 371 Date: Mar. 18, 2014

(30) Foreign Application Priority Data
Jul. 28, 2011 (FR) 1156897

(51) Int. Cl. A61B 10/02; A61B 10/00; A01K 11/00

(52) U.S. Cl. CPC A61B 10/0266; A01K 11/00; A61B 10/0096
USPC 600/564; 29/428

(57) ABSTRACT

The invention relates to a system for collecting at least one sample of animal tissue, including: a sampling device, including at least one cutting element for cutting off a sample of animal tissue, and a storage device including a receiving tube for receiving said sample. According to the invention, the system includes a weighted element (22) configured so as to assume at least two positions, i.e. a first position which is assumed prior to collecting said sample and in which said weighted element (22) is retained in said sampling device or in said storage device, and a second position which is assumed after said sample is collect and in which said weighted element (22) is free within said tube, said weighted element (22) being pushed inside said tube during said sampling.

### 1. FIELD OF THE INVENTION

The field of the invention is that of the control and/or identification of animals.

More specifically, the invention pertains to the collecting and storing of animal tissue, making it possible especially to preserve cells carrying the animal's biological or biochemical characteristics, for example in order to subsequently identify the animal or detect sickness in the animal. It can be noted that such collecting can be done on any animal species (cattle, sheep, pigs, goats, poultry, fish, etc.) with or without the simultaneous placing of an identification tag.

Even more specifically, the invention pertains to the means for storing and preserving such a collected sample.

### 2. PRIOR ART

In order to improve the tracking of livestock, improve productivity (by eliminating diseased animals or by searching for singular genetic characteristics for example) and/or guarantee the origin of animals intended especially for consumption, for example by detecting illnesses, it is increasingly becoming common practice to carry out one or more operations for collecting tissues from the animals concerned.

Such collecting can be done on the animal when placing an identification tag on the animal, at birth for example, or subsequently throughout the animal's existence. Several sample-collecting operations can thus be done, especially in order to detect diseases or certify the animal's identity by a comparison of DNA sequences.

Tissue-collecting techniques classically implemented to collect a sample of tissue independently of the placing of an identification tag, rely for example on the use of a punch forming or comprising a cutting element, having a generally circular cutting edge (that works by continuous contact or is serrated), whose purpose is to cut out a sample of animal tissue and collect it in a housing.

As illustrated in FIG. 1, the punch 10 is classically fixed to a first jaw of a tool for collecting, a set of pliers for example. A microtube 12, intended for storing the sample after it is collected, is fixed to a second jaw of the tool. Such a microtube 12 is classically closed by an seal before the sample is collected, especially to protect the interior of the microtube 12 from the outside environment.

When the tool is actuated, the cutting element 10 cuts out the animal's skin, tears the seal serving as a lid for the microtube 12, and gets inserted at least partially into the microtube 12. The punch 10 therefore conventionally has a diameter slightly smaller than that of the microtube 12 so as to serve as a plug for the microtube.

Once collected, the sample of animal tissue can be stored and/or forwarded to a laboratory for analysis.

Through this collecting technique, the sample 11 is directly inserted into the microtube 12 after perforation of the seal, thus limiting the risk of contamination of the sample.

However, one drawback of this technique is that, after collecting, the collected sample remains generally wedged in the housing provided for this purpose in the cutting element.

The punch must then be withdrawn or cut out carefully during subsequent analysis of the sample in order to avert the loss or contamination of the sample.

The operation of extracting the sample of tissue collected in the housing, thus proves to be difficult and/or complicated and presents a risk of loss of the sample as well as a risk of inflicting cuts on the user.

In addition, the preservation of the sample in the cutting element is not done optimally

There is therefore need for a novel technique for collecting tissue from an animal that does not have all these prior-art drawbacks.

### 3. SUMMARY OF THE INVENTION

The invention proposes a novel solution that does not have all these drawbacks of the prior art, in the form of a system for collecting at least one sample of animal tissue, comprising:
a device for collecting comprising at least one cutting element designed to cut out a sample of animal tissue, and
a device for storing comprising a receiving tube designed to receive the sample.

According to the invention, such a system also comprises a weighted element configured to take at least two positions, comprising one first position prior to the collecting of the sample, in which the weighted element is held in the device for collecting or in the device for storing, and a second position following the collecting of the sample, in which the weighted element is released in the tube, the weighted element being pushed within the tube during the collecting.

In particular, the weighted element is a sphere or a cylinder having a density greater than or equal to that of at least one specific agent contained in the tube.

Thus, the invention relies on the use of a novel element, original in collecting systems, which, in a first position, is fixedly attached in the device for collecting or in the device for storing and, in a second position, is unattached in the device for storing, capable of fulfilling several functions according to the position that it occupies.

For example, in the first position, the weighted element is held in the upper part of the device for storing and shuts off an inlet orifice of the tube. In this position, prior to collecting, corresponding for example to the shape in which the receiving tubes are commercially distributed, the weighted element fulfils the function of a plug of the receiving tube, making it possible to prevent the introduction of impurities into the tube and therefore the contamination of the interior of the tube by the external surroundings.

Should the tube contain at least one specific agent of the following types: preserving agent, desiccant, reagent, sample-preparing agent, etc, the plug-forming weighted element will prevent the loss of the specific agent (which can especially take the form of a gel, a cream, an oil, a liquid, a powder, a gas, an impregnated foam, etc) in ensuring the tight-sealing quality of the tube before it is used.

In particular, an agent of this kind enhances the preservation of the sample, to prepare it for subsequent treatment such as DNA analysis and/or to treat it directly. In other words, the specific agent can take the form of any product capable of acting on a sample of tissue collected from the animal.

According to another example, in the first position, the weighted element is kept in the device for collecting, within the cutting element.

In the second position subsequent to the collecting, the weighted element is released in the sample tube.

When the tube contains at least one specific agent (inserted in the tube prior to the collecting, for example during the manufacture of the tube, or inserted in the tube after the collecting, for example during the analysis of the sample in the laboratory), the weighted element is in contact with the specific agent. It can then fulfill the function of a stirrer or mixer, enabling especially the distribution of the specific agent throughout the sample, for example by mixing and homogenizing the liquid if the specific agent is present in liquid form. It can also act as ballast, enabling the sample to be made to sink to the bottom of the tube or again as a “pestle” used to crush or break up the sample in order to facilitate subsequent analysis.

In particular, the weighted element takes the form of a sphere (a bead) or a cylinder.

In this way, it has a circular section adapted to the inlet orifice of the tubes. Furthermore, it can be inserted into the inlet orifice of a tube whatever its orientation.

In particular, the weighted element has a diameter substantially equal to the diameter of the inlet orifice of the tube or, should the tube be provided with a tube head (tube top piece), a diameter appreciably equal to the diameter of the central aperture of the tube head and greater than the diameter of an inlet orifice of a pipette that is to extract the sample from the tube or a certain quantity of specific agent.

Thus, the weighted element enables the hermetic or almost-hermetic closure of the tube before its use (i.e. in its first position, when it is provided in the device for storing). Furthermore, the weighted element does not close off the pipettes used during the pipette operation, its size being adapted to prevent any suction in the pipette. Besides, the density of the weighted element enables it to sink to the bottom of the tube in its second position. It therefore does not obstruct the pipette by contact.

In particular, the weighted element has a density greater than or equal to that of the specific agent.

For example, it has a density greater than that of the liquid or liquids used for the preservation of the collected tissues and/or that of the liquids used during subsequent analyses.

In this way, in the second position, the weighted element “falls” to the bottom of the tube, possibly carrying the sample along with it.

Thus, the weighted element can especially facilitate the mixing of reagents in the tube.

According to one particular characteristic of the invention, the weighted element has a specific color.

Such a color is preferably chosen to be bright or striking, for example apple green. The weighted element, in its second position, can then serve as a visual indicator of efficient collecting. Indeed, once the collecting has been made, the weighted element “falls” to the bottom of the tube, possibly dragging along the sample, and the presence of the weighted element, with its bright color, in the tube can be checked.

As the case may be, the color of the weighted element can be modified in contact with the specific agent and/or the cutting element.

According to one variant of the invention, the weighted element comprises means for hooking or holding on to the sample, enabling the sample to be fixedly attached to the weighted element.

For example, such means for hooking comprise an element belonging to the following group:
a spike;
a needle;
a hook;
a harpoon.

Thus, during the collecting of the tissues, the sample is pushed against the weighted element and gets hooked onto the weighted element. In its second position, the weighted element drives the sample in the tube, advantageously to the bottom of the tube.

It can be seen in this way that the sample collected is in contact with the specific agent, thus enabling better preservation of the sample of tissue. Furthermore, this variant restricts the quantity of agent needed for the reaction, since a small quantity of agent is sufficient to cover the sample if it has “sunk” to the bottom of the tube.

According to another aspect of the invention, the weighted element is inert.

The term “inert” or “neutral” is understood to refer to a material that does not break down and produces no physical or chemical reaction. In other words, the weighted element does not deteriorate in contact with other materials such as animal tissues or the specific agent. Furthermore, it is chemically compatible with the specific agents used.

In another embodiment, the weighted element may be not inert and capable of reacting with the sample and/or a specific agent provided in the tube (introduced before, during or after the collecting).

As a variant, the weighted element can have an inert “core” and be covered with a reagent or, on the contrary, it can have a non-inert core capable of containing such a reagent. In other words, the weighted element can be formed “partially” by an inert material.

According to yet another aspect, the weighted element comprises a magnet.

In this way, the weighted element comprises a magnetic material and can go into motion when it is subjected to a magnetic field, a hot plate, etc. It can then mix the content of the tube to favor the reagents between the specific agent and the tissue sample.

Naturally, other materials could be used for the weighted element such as glass, or steel for example.

In one particular embodiment, the device for storing comprises a tube head comprising a hood pierced with a central aperture and a flange, capable of taking support on the rim of the tube. In the first position, the weighted element can then close off the central aperture.

The use of a tube head brings numerous advantages. First of all, it can provide a support on which a cutting element can take support in order to accurately cut out the tissues of the animal. It can also enable the closing of the tube, for example by the tight-fitting or clipping, into the tube head, of the cutting element or a pusher element as defined in the patent application WO2010/066475 filed on 31 Jul. 2009 on behalf of the same Applicant. In addition, the presence of such a tube head enable the opening of the tubes by the analysis laboratories to be automated through the decapsulation of the tube head in such a way that only the sample and the weighted element remain within the tube.

According to yet another characteristic, the device for collecting comprises a pusher element that is mobile relative to the cutting element making it possible to push the sample into the device for storing after the sample has been cut out by the cutting element, the weighted element being pushed by the pusher element into the tube during the collecting.

In particular, according to the present technique, as described in the patent application WO2010/066475 mentioned here above, the pusher element pushes the sample, tears a lid closing the tube and closes the tube. According to this embodiment of the invention, if the weighted element is held in the device for storing in its first position, the pusher element pushes the sample into the inlet orifice of the tube or the central aperture of the tube head which in turn pushes the weighted element into the tube and then the pusher element closes the tube. If the weighted element is held in the device for collecting in its first position, the pusher element pushes the weighted element which in turn pushes the sample into the inlet orifice of the tube or the central aperture of the tube head and then the pusher element closes the tube.

Such a collecting system thus has improved tight-sealing quality as compared with the prior-art techniques and the industrial-scale manufacture and laboratory processing of the tube obtained after collecting are optimized.

In another embodiment, the invention pertains to a device for collecting of a collecting system as described here above, comprising a weighted element.

More specifically, such a device for collecting at least one sample of animal tissue comprises at least one cutting element intended to cut out a sample of animal tissue and is designed to cooperate with a device for storing comprising a receiving tube intended for receiving the sample.

According to the invention, such a device for collecting comprises a weighted element in a first position prior to the collecting of the sample, in which the weighted element is held in the device for collecting, the weighted element being a sphere or a cylinder having a density greater than or equal to that of at least one specific agent contained in the tube. The weighted element is configured to take at least one second position after the collecting of the sample, in which the weighted element is released in the receiving tube, the weighted element being pushed inside the tube during the collecting.

In yet another embodiment, the invention pertains to a device for storing of a collecting system as described here above, comprising a weighted element.

More specifically, such a device for storing at least one sample of animal tissue comprises a receiving tube to receive a sample of animal tissue and is designed to cooperate with a device for collecting comprising at least one cutting element intended for cutting out the sample.

According to the invention, such a device for storing comprises a weighted element in at least one first position prior to the collecting of the sample, in which the weighted element is held in the device for storing, the weighted element being a sphere or a cylinder having a density greater than or equal to that of at least one specific agent contained in the tube. The weighted element is configured to take at least one second position after the collecting of the sample, in which the weighted element is released in the receiving tube, the weighted element being pushed inside the tube during said collecting.

Such a device for collecting and/or device for storing has the same advantages as the collecting system described here above. They are not described in more ample detail.

The invention also pertains to a method for manufacturing a device for collecting or a device for storing as described here above comprising a step for inserting a weighted element into the device for collecting or into the device for storing. Such a weighted element is configured to take at least two positions, including a first position prior to the collecting of the sample in which the weighted element is held in the device for collecting or in the device for storing and a second position after the collecting of the sample, in which the weighted element is released in the tube, the weighted element being pushed into the tube during the collecting. The weighted element is a sphere or a cylinder having a density greater than or equal to that of at least one specific agent contained in the receiving tube.

In particular, when the weighted element is inserted into the device for storing, the step for inserting implements a closing-off of an inlet orifice of the tube.

Again, the advantages of this method of manufacture are the same as those presented with reference to the system for collecting. They are not described in more ample detail. In particular, such a method of manufacture is simple to implement and easily applicable at an industrial scale.

According to one variant, such a method of manufacture comprises a first step for the insertion, into the tube, of a tube head comprising a hood pierced with a central aperture and a flange, designed to take support on the rim of the tube and a second step for the insertion, into the tube head, of the weighted element in such a way that, in the first position, the weighted element closes off the central aperture of the tube head.

The insertion of the weighted element into the tube head after this tube head has been placed into a tube advantageously makes it possible to reduce the pressure of the tube in compressing a smaller volume of air.

### 4. LIST OF FIGURES

Other features and advantages of the invention shall appear more clearly from the following description of a particular embodiment, given by way of a simple illustratory and non-exhaustive example and from the appended figures, of which:

FIG. 1 presented with reference to the prior art illustrates a device for collecting a sample of tissue according to the prior art;

FIGS. 2A to 2C illustrate the first embodiment of the invention, according to which the weighted element is, in its first position, held in a receiving tube of a device for storing;

FIGS. 3A to 3C present a second embodiment of the invention, according to which the weighted element is, in a first position, held in the tube head of a receiving tube of a device for storing;

FIGS. 4A to 4C illustrate a third embodiment of the invention in which the weighted element is, in its first position, held in a device for collecting;

FIG. 5 illustrates a system of collecting according to the second embodiment;

FIG. 6 presents the main steps implemented by a manufacturing device according to the invention.

### 5. DESCRIPTION OF ONE EMBODIMENT OF THE INVENTION

### 5.1 General Principle

The general principle of the invention relies on the use of a weighted element combined with the use of a device for collecting and/or storing to form a system for collecting a sample of animal tissues in a particularly ingenious manner. Here, the term “weighted element” is understood to mean an element having a certain mass.

The invention thus proposes a novel system of collecting in which there is inserted the weighted element that can take at least two positions.

In particular, in a first position prior to the collecting of the sample, the weighted element is held in position in the device for collecting or the device for storing. Advantageously, when it is held in the device for storing, the weighted element closes off the inlet orifice of the tube and can fulfill a function of a plug of the receiving tube.

In a second position, subsequent to the collecting of the sample, the weighted element is introduced into the tube at the same time as the sample and released inside the tube. It can therefore fulfill a function of stirrer or mixer, of ballast (or weight) or of pestle.

Thus, in one advantageous embodiment, the weighted element comprises a magnetic element used to fulfill the function of a stirrer in the receiving tube when it takes a second position. The weighted element then facilitates the mixing in the tube especially when the reagents are added into the tube during the analysis of the sample.

It can be noted however, that the weighted element is preferably made out of a neutral and inert material, i.e. a material that produces no chemical or physical reaction with specific reagents or agents introduced into the receiving tube before or after the collecting.

Besides, the weighted element advantageously takes the form of a sphere or a cylinder having a circular section to adapt as efficiently as possible to the usual shape of receiving tubes. Thus, it preferably has a diameter substantially equal to (or very slightly smaller than) the diameter of the inlet orifice of the tube. Preferably, its diameter is also greater than the diameter of the inlet pipette of a pipette used to extract the sample from the tube or a quantity of specific agent.

### 5.2 First Embodiment

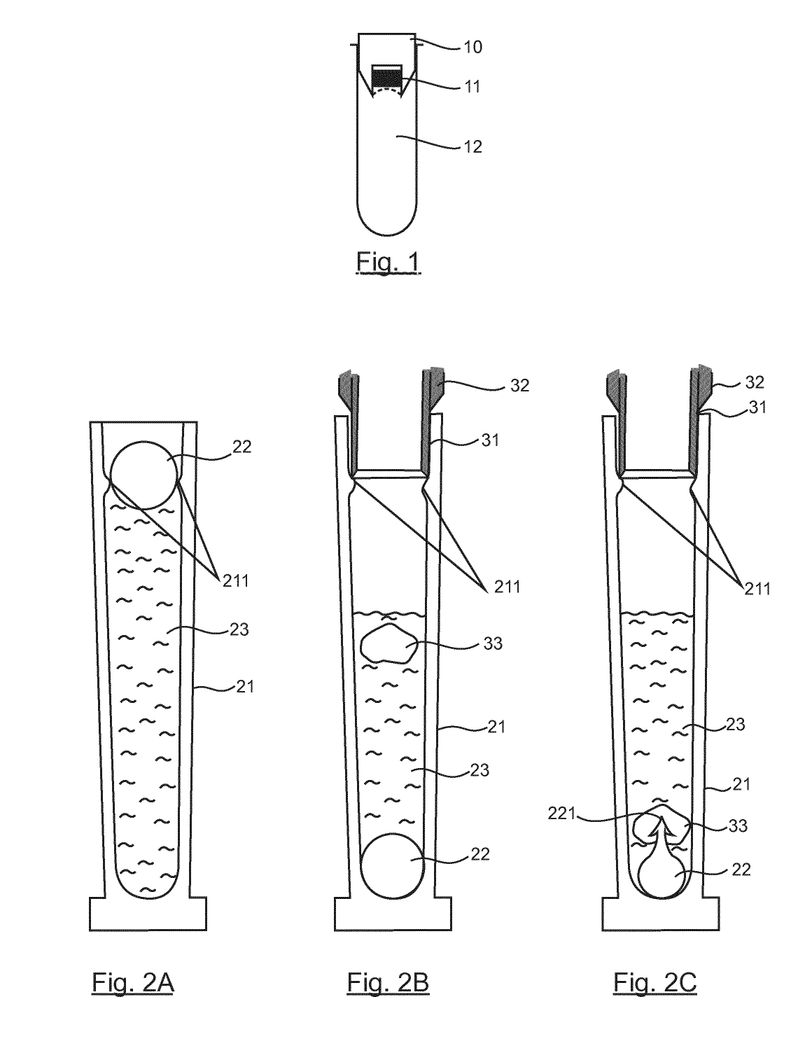

FIGS. 2A to 2C illustrate a first embodiment of the invention in which the weighted element is present in the device for storing and directly held in the receiving tube, prior to the collecting.

More specifically, FIGS. 2A to 2C present a view in section of a collecting system according to this first embodiment, comprising:
a device for collecting comprising a cutting element 31 that is designed to cut out a sample of animal tissue, and
a device for storing comprising a receiving tube 21 that is designed to receive the sample and a weighted element 22, for example a hollow or solid sphere, also called a bead or a ball.

The shape of the receiving tube 21 is considered for example to be compatible with a support of sample tubes of a rack type comprising for example 24, 48 or 96 positions.

According to a first example of an embodiment, the receiving tube 21 is considered to contain, prior to the collecting, at least one specific agent 23 in the form of a gel, a liquid, a powder, beads or foam making it possible especially to improve the preservation of the sample or to prepare it for future analyses in the laboratory. Naturally, the receiving tube can be empty before the collecting and a specific agent of this kind can be introduced after the collecting, by the person carrying out the collecting or by a person analyzing the collected sample for example.

Initially, referring to FIG. 2A, we describe the first position of the weighted element 22 prior to the collecting operation.

In this first position, the weighted element 22 is held in position in the upper part of the device for storing, at the inlet of the receiving tube 21, by pins or stops 211 present on the internal surface of the tube, and closes off the inlet orifice of the tube. Thus, the weighted element 22 serves as a plug for the receiving tube 21 and the tight sealing of the specific agent 23 present in the receiving tube 21 is ensured.

Referring now to FIG. 2B, we describe the second position of the weighted element 22 after collecting of the sample.

As illustrated in this figure, during the collecting, the cutting element 31 of the weighted element possibly fixedly attached to the support 32 cuts out the animal's skin, pushes the weighted element 22 into the tube 21 and gets inserted at least partially into the tube 21. The cutting edge of the cutting element 31, which has a diameter slightly smaller than that of the inlet hole of the receiving tube so as to be capable of getting inserted in the tube 21, then comes into a position where it abuts the pins 211 present on the inner surface of the tube. For this purpose, the weighted element 22 and/or the walls of the tube 21 are considered to be capable of underoing slight deformation.

It may be recalled that the support 32 which can be made out of plastic, is conventionally mounted detachably at the end of a push-rod fixedly attached to one of the jaws of a set of collecting pliers. It takes the form of a surface generated by a revolution having the same axis as the cutting element 31. According to one variant, the cutting element 31 and the support 32 are formed as one piece, for example out of plastic or metal. The cutting element 31 and the support 32 forming a single piece are then considered to be a monoblock unit.

The weighted element 22 is then released in the tube 21 between the bottom of the tube and the sample 33. In particular, the weighted element 22 has a density greater than that of the specific agent 23, and sinks to the bottom of the receiving tube 21 when this tube is immobile. The weighted element 22, in its second position, can then fulfill the function of a stirrer or mixer. Furthermore, the weighted element can serve as a visual indicator to indicate that the collecting has been properly done, especially if it has a bright color.

FIG. 2C illustrates one variant of an embodiment of the weighted element.

In this variant, the weighted element 22 has a means 221 for hooking the sample 33, enabling the sample to be fixedly attached to the weighted element during the collecting and to pull it into the bottom of the receiving tube 21. This variant also makes it possible to provide for better preservation and/or better treatment of the sample by the specific agent 23. Indeed, in this way the sample 33 has a high probability of being covered by the specific agent (in liquid form for example) without in any way thereby requiring a major quantity of this agent.

Different forms have been envisaged for this hooking means 221 of the sample.

Thus, as illustrated in FIG. 2C, the hooking means 221 take the form of a harpoon. According to other variants, not shown, such hooking means take the form of a spike, a needle, a hook, etc.

### 5.3 Second Embodiment

FIGS. 3A to 3C illustrate a second embodiment of the invention in which the weighted element is present in the device for storing and held in a tube head of the receiving tube prior to the collecting.

More specifically, FIGS. 3A to 3C present a view in section of a device for storing according to this second embodiment comprising a receiving tube 25 intended to receive the sample, a tube head 24 and a weighted element 22, for example a hollow or solid sphere, also called a bead or a ball.

Such a tube head 24 is fixedly attached to the inlet of the receiving tube 25 for example by being clipped on or fitted in. It can be made out of a flexible material, especially rubber, plastic material or the like, to facilitate its insertion into the neck of the tube.

More specifically, the tube head 24 takes the form of a hood pierced with a central aperture with a diameter sufficient to enable the insertion of the weighted element 22. The use of flexible material for this hood also facilitates the setting up of the weighted element. The tube head 24 also has a flange capable of being supported on the rim of the receiving tube 25. The use of such a flange makes it possible especially to facilitate the laying and removal of the hood. The flange also defines an abutment surface on which the cutting edge of a cutting element can take support during the collecting in order to cut out the sample of tissue more easily. It can be noted that the tube can also be made out of a more or less elastic material. However, the fact is that it is desirable for the tube head to be rigid enough to serve as a supporting surface for an efficient cutting out of the sample.

Once again, the shape of the receiving tube 25 is considered to be compatible with a rack type of sample tube carrier comprising for example 24, 48 or 96 positions.

First of all, referring to FIG. 3A, a description is provided of the first position of the weighted element 22 prior to the collecting.

In this first position, the weighted element 22 is introduced into the tube head 24 so as to plug the central aperture of the tube head 24 of the tube 25 which, as described with reference to the first embodiment, may or may not contain a specific agent 23. For example, the weighted element is force-fitted into the tube head and held in the tube head through the elastic properties of the material used for the tube head. Again, as described with reference to the first embodiment, the weighted element 22 serves as a plug or lid for the receiving tube 25 in this first position.

Here below, referring to FIG. 3B, we describe the second position of the weighted element 22 after collecting of the sample.

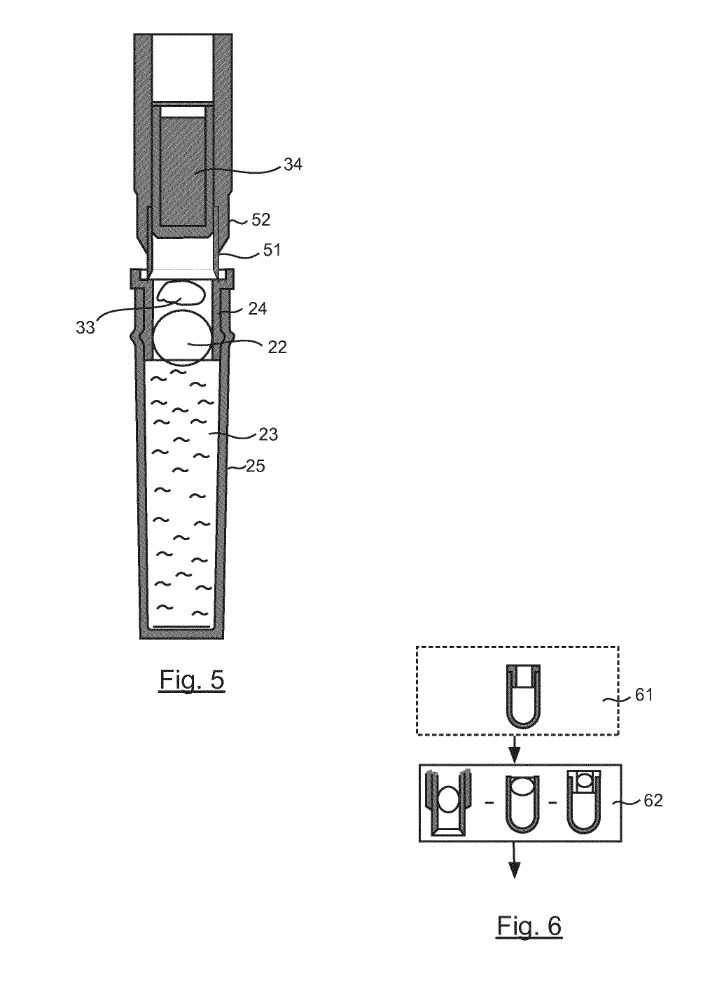

For example, as illustrated in FIG. 5, it is considered that the collecting implements the means for collecting described in the patent application WO2010/066475 comprising two distinct elements that are mobile relative to each other. Of these two distinct elements, a cutting element 51 enables the cutting out of the tissues of the animal and a pusher element 34 enables the sample thus cut out to be pushed into the device for storing. Naturally, a conventional collecting device could also be used with a device for storing according to the second embodiment.

As illustrated in this FIG. 5, during the collecting, the cutting element 51, which is possibly fixedly attached to a support 52, initially cuts out the animal's skin.

In a second stage, illustrated in FIG. 3B, the pusher 34 pushes the sample 33 which has thus been cut into the channel of the tube head 24 through its central aperture, pushes the weighted element 22 into the tube 25 and gets at least partially inserted into the tube head 24. The pusher 34 is then fixedly attached to the tube head 24 by force-fitting for example and thus hermetically or almost hermetically closes off the tube 25.

The weighted element 22 is then released into the tube 25 between the bottom of the tube and the sample 23 in its second position. In particular, as described with reference to the first embodiment, if the weighted element 22 has a density greater than that of the specific agent 23, it sinks to the bottom of the receiving tube 25 when this tube is immobile. The weighted element 22, in its second position, can then fulfill a stirrer or mixer function.

Once the sample has been collected and the pusher 34 at least partially inserted into the central aperture of the tube head 24, it is possible, by grasping the flange, to remove the tube head 24 and the pusher element 34 as a single unit in order to analyze the sample. It is thus possible to automate this step for opening the receiving tube 25, enabling the tube head to be decapsulated so as to leave only the tissue sample in the tube 25.

FIG. 3C illustrates an alternative embodiment of the weighted element 22.

In this variant, as described with reference to FIG. 2C, the weighted element 22 has means 221 for hooking the sample 33, taking the form of a harpoon, a spike, a needle, a hook, etc., used to fixedly attach the sample to the weighted element during the collecting and to carry it into the bottom of the receiving tube 51.

This variant makes it possible to provide for better preservation and/or processing of the sample by the specific agent 23 provided in the tube prior to the collecting or introduced subsequently.

### 5.4 Third Embodiment

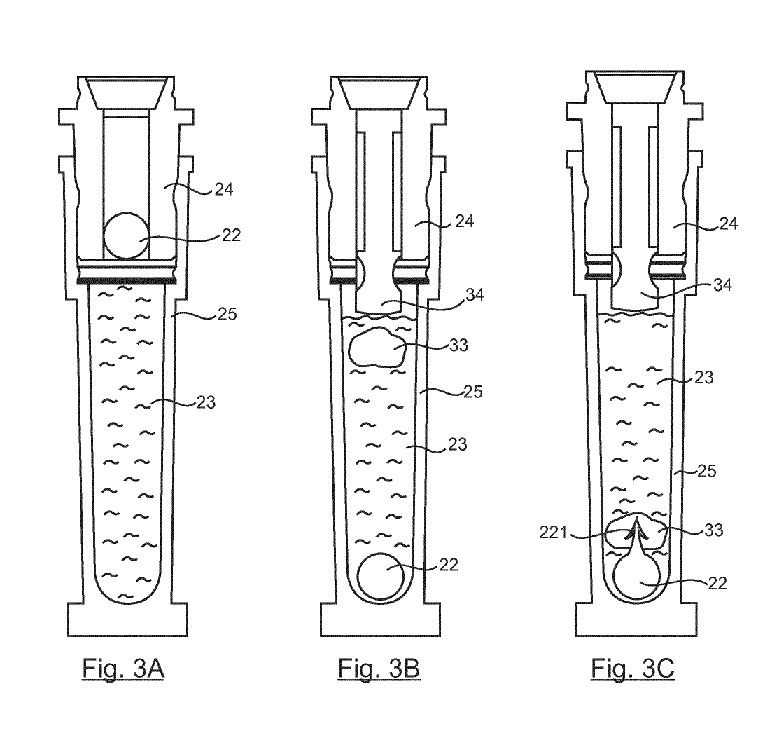

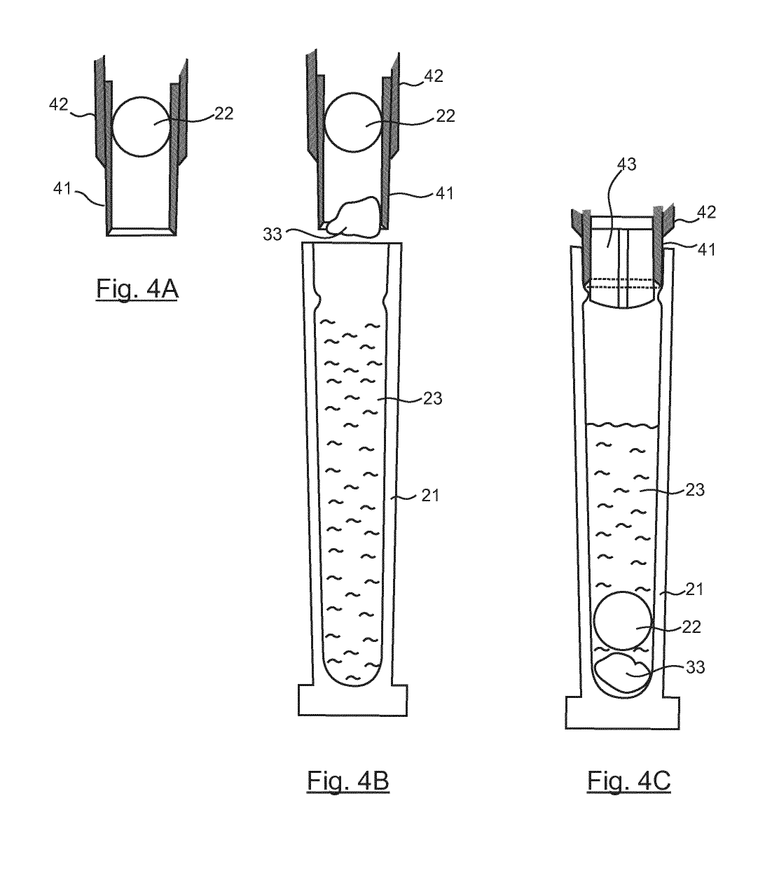

FIGS. 4A to 4C illustrates a third embodiment of the invention in which the weighted element is present in the device for collecting and held in the cutting element or in a support of the cutting element prior to the collecting.

More specifically, the steps 4A to 4C present a view in section of a collecting system in this third embodiment which comprises:
a device for collecting comprising a cutting element 41 intended to cut out a sample of animal tissue and a weighted element 22, for example a hollow or solid sphere also called a ball;
a device for storing comprising a receiving tube 21 intended to receive the sample.

Firstly, referring to FIG. 4A, a description is provided of the first position of the weighted element 22, prior to the collecting.

In this first position, the weighted element 22 is introduced into the device for collecting, for example inside the cutting element 41 or a support 42 of the cutting element (which generally takes a shape generated by revolution).

For example, the weighted element is force-fitted into the device for collecting and held in position through the mechanical properties of the weighted element or of the collecting device. According to one variant, not illustrated, the weighted element 22 is kept in its first position by pins or stops present on the internal surface of the cutting element 41 or its support 42.

As illustrated with reference to FIG. 4B, during the collecting operation, the cutting element 41 initially cuts out the animal's skin.

In a second stage, illustrated in FIG. 4C, the sample is pushed out of the device for collecting, using the weighted element 22. In other words, an extractor 43, for example of a needle, rod, or pusher element 34, as described with reference to FIGS. 3A to 3C, type etc., sliding in the cutting element 41, pushes the weighted element 22 towards the tube 21, the weighted element in turn carrying along the sample 33 into the sample tube 21. The weighted element 22 is then released in the tube 21 in its second position.

Owing to the specific properties of the weighted element 22, especially its weight, the weighted element 22 is driven towards the bottom of the tube 21 and, with it, pulls along a sample 33. The weighted element, in its second position, can then fulfill a ballast or weighting function for the sample 33 or a pestle function enabling a crushing or breaking up of the sample in order to facilitate subsequent analysis.

It can be noted that, according to one variant, such a weighted element 22 can be equipped with a means for hooking the sample as illustrated in FIGS. 2C and 3C, enabling the sample to be fixedly attached to the weighted element.

The extractor 43 can be actuated by hand or by means of a dual-motion collecting tool (one actuating the cutting element and the other the extractor).

This third embodiment ensures that the sample 33 is situated between the bottom of the tube 21 and the weighted element 22 after the collecting operation. Thus, it is ensured that there is a better preservation and/or a better processing of the sample by a specific agent 23 which can be provided in the tube prior to the collecting or introduced subsequently to the collecting.

Naturally, this third embodiment can be implemented with a receiving tube which may or may not comprise a tube head. In the same way, it can be implemented in a device for collecting as described in the patent application WO2010/066475 mentioned here above, comprising distinct elements that are mobile relative to one another.

### 5.5 Method of Manufacture

Here below, referring to FIG. 6, we present the main steps of manufacture of a collecting system comprising a device for collecting and/or a device for storing according to the embodiments described here above.

According to these embodiments, such a method comprises a step 62 for inserting a weighted element into the device for collecting or into the device for storing, said weighted element being configured to take the two positions described further above.

Such a step can be preceded by a step 61 for inserting a tube head into the receiving tube. In this case, the weighted element can be inserted into the tube head once the tube head itself has been inserted into the receiving tube in order to create less pressure in the receiving tube by compressing a smaller volume of air.

1. System for collecting at least one sample (33) of animal tissue, comprising:
a device for collecting comprising at least one cutting element (31; 41; 51) designed for cutting out a sample of animal tissue, and
a device for storing comprising a receiving tube (21; 25) designed for receiving said sample,
wherein said system also comprises a weighted element (22) configured to take at least two positions, comprising one first position prior to the collecting of said sample, in which said weighted element (22) is held in said device for collecting or in said device for storing, and a second position subsequent to the collecting of said sample, in which said weighted element (22) (22) is released in said tube, said weighted element (22) being pushed into said tube during said collecting,
and wherein said weighted element (22) is a sphere or a cylinder having a density greater than or equal to that of at least one specific agent (23) contained in said tube (21; 25).

2. System for collecting according to claim 1, wherein said at least one specific agent belongs to the group comprising:
a preserving agent,
a desiccant,
a reagent,
an agent for preparing said sample.

3. System for collecting according to any one of the claims 1 and 2, wherein said weighted element (22) has a specific color.

4. System for collecting according to any one of the claims 1 to 3, wherein said weighted element (22) comprises means (221) for hooking on to said sample, enabling the sample (33) to be fixedly attached to said weighted element (22)

5. System for collecting according to claim 4, wherein said means for hooking (221) comprise an element belonging to the group comprising:
a spike;
a needle;
a hook;
a harpoon.

6. System for collecting according to any one of the claims 1 to 5, wherein said weighted element (22) is inert.

7. System for collecting according to any one of the claims 1 to 6 wherein said weighted element (22) comprises a magnet.

8. System for collecting according to any one of the claims 1 to 7, wherein, in said first position, said weighted element (22) is held in the upper part of said device for storing, and closes off an inlet orifice of said tube (21; 25).

9. System for collecting according to claim 8 characterized in said device for storing comprises a tube head (24) comprising a hood, pierced with a central aperture, and a flange, and wherein, in said first position, said weighted element (22) closes off said central aperture.

10. System for collecting according to any one of the claims 1 to 7, wherein, in said first position, said weighted element (22) is held in said device for collecting, inside said cutting element (41).

11. System for collecting according to any one of the claims 1 to 10, wherein said device for collecting also comprises a pusher element (34) that is mobile relative to said cutting element, configured to push said sample into said device for storing after the sample has been cut out by said cutting element,
said weighted element being pushed by said pusher element into said tube during said collecting.

12. Device for collecting at least one sample of animal tissue comprising at least one cutting element (31; 41; 51) for cutting out a sample of animal tissue,
designed to cooperate with a device for storing comprising a receiving tube (21; 25) designed for receiving said sample.
wherein said device for collecting comprises a weighted element (22) in a first position prior to the collecting of said sample, in which said weighted element (22) is held in said device for collecting,
and wherein said weighted element (22) is a sphere or a cylinder having a density greater than or equal to that of at least one specific agent (23) contained in said tube (21, 25), said weighted element (22) being configured to take at least one second position subsequently to the collecting of said sample, in which said weighted element (22) is released in said receiving tube, the weighted element (22) being pushed into the tube during said collecting (21, 23).

13. Device for storing at least one sample of animal tissue comprises a receiving tube (21; 25) for receiving a sample of animal tissue,
designed to cooperate with a device for collecting comprising at least one cutting element designed (31; 34; 51) for cutting out said sample.
wherein said device for storing comprises a weighted element (22) in a first position prior to the collecting of said sample, in which said weighted element (22) is held in said device for storing,
and wherein said weighted element (22) is a sphere or a cylinder having a density greater than or equal to that of at least one specific agent (23) contained in said tube (21; 25), said weighted element being configured to take at least one second position subsequently to the collecting of said sample, in which said weighted element (22) is released in said receiving tube (21; 25),
said weighted element (22) being pushed into said tube during said collecting.

14. Method for manufacturing a device for collecting according to claim 12 or a device for storing according to claim 13,
wherein it comprises a step for inserting a weighted element (22) into said device for collecting or into said device for storing,
said weighted element (22) being a sphere or a cylinder having a density greater than or equal to that of at least one specific agent (23) contained in said tube (21; 25),
said weighted element (22) being configured to take at least two positions, comprising a first position prior to the collecting of said sample in which said weighted element (22) is held in said device for collecting or in said device for storing and a second position subsequent to the collecting of said sample, in which said weighted element (22) is released in said tube, said weighted element (22) being pushed into said tube during said collecting.
